(19) United States
(12) Patent Application Publication
(10) Pub. No.: US 2021/0155885 A1
(43) Pub. Date: May 27, 2021

(54) Bioreactors

(71) Applicant: Global Life Sciences Solutions USA LLC, Marlborough, MA (US)

(72) Inventor: Ralph Stankowski, Westborough, MA (US)

(21) Appl. No.: 17/047,864

(22) PCT Filed: Apr. 25, 2019
(86) PCT No.: PCT/US2019/029131

§ 371 Date: Oct. 15, 2020

Related U.S. Application Data
(60) Provisional application No. 62/662,304, filed on Apr. 25, 2018.

(51) Int. Cl. C12M 1/24; C12M 1/00

(52) U.S. Cl. CPC C12M 23/08; C12M 23/22; C12M 27/20

(57) ABSTRACT

Disclosed are bioreactor flasks (10) including a volume extending between a first volume end and a second volume end for the cultivation of cells or other microorganisms, said volume having a horizontal cross section area (CSA) which increases in a direction from the first volume end to the second volume end. The flask optionally includes a housing including a cylindrical lower portion (14) and an inverted truncated conical upper portion (16) which provides said increasing CSA. Disclosed also are arrays of flasks (10), supported in a tray for collective agitation.

### CROSS-REFERENCE TO RELATED APPLICATIONS

This application claims the benefit of U.S. Provisional Application No. 62/662,304, filed Apr. 25, 2018, which is incorporated herein by reference in its entirety.

### TECHNICAL FIELD

The present disclosure relates to bioreactors, in particular, bioreactor flasks for cultivation of cells or other biological microorganisms.

### BACKGROUND

The bio-processing industry has traditionally used stainless steel systems and piping in manufacturing processes for fermentation and cell culture. These devices are designed to be steam sterilized and reused. Cleaning and sterilization are however costly labour-intensive operations. Moreover, the installed cost of these traditional systems with the requisite piping and utilities is often prohibitive. Furthermore, these systems are typically designed for a specific process, and cannot be easily reconfigured for new applications. These limitations have led to adoption of a new approach in recent years—that of using plastic, single-use disposable bags and tubing, to replace the usual stainless steel tanks.

In particular bioreactors, traditionally made of stainless steel, have been replaced in many applications by disposable bags (cell bags) which are rocked to provide the necessary aeration and mixing necessary for cell culture. These single-use bags are typically provided sterile and eliminate the costly and time-consuming steps of cleaning and sterilization. The bags are designed to maintain a sterile environment during operation thereby minimizing the risk of contamination.

Commonly used bags are of the “pillow style,” mainly because these can be manufactured at low cost by seaming together two flexible sheets of plastic. Three-dimensional bags have also been described, where further sheets may be used to create wall structures.

Such cell bags have disadvantages, when initial low volumes of cells, known as seed volumes are introduced into the bag. Further, to arrive at that seed volume, it is currently often necessary to transfer a small vial of cells suspended in about 1-2 ml of liquid multiple times into different vessels to multiply the cells sufficiently to provide a viable seed volume, thereby increasing costs and the risk of contamination. The footprint of the equipment required is excessive for a small laboratory.

### SUMMARY

Devices and assemblies according to the present disclosure address the problems mentioned above by reducing the number of transfer stages required for a seed volume by means of a bioreactor flask having a horizontal cross sectional area (CSA) which increases vertically and optionally having internal detail to improve cell growth.

In some embodiments, a bioreactor flask includes a volume extending between a first volume end and a second volume end for the cultivation of cells or other microorganisms. The volume has a horizontal cross section area (CSA) which increases in a direction from the first volume end to the second volume end. In some embodiments, the second volume end is gravitationally upward relative to the first volume end.

With the above mentioned flask a very small initial volume of seed cell can be introduced into a acceptably small volume, and as the cells divide and multiply, their increasing culture volume can be accommodated by virtue of the vertically increasing CSA, without having to transfer the cells to an increased volume vessel.

The present disclosure extends to any combination of features disclosed herein, whether or not such a combination is mentioned explicitly herein. Further, where two or more features are mentioned in combination, it is intended that such features may be claimed separately without extending the scope of the invention.

### BRIEF DESCRIPTION OF THE DRAWINGS

The disclosure can be put into effect in numerous ways, illustrative embodiments of which are described below with reference to the drawings, wherein:

FIG. 1 shows one embodiment of a bioreactor flask according to the invention;

FIG. 2 shows a second embodiment of a bioreactor flask according to the invention;

FIG. 3 shows an array of bioreactor flasks of the type shown in FIG. 1; and

FIG. 4 shows an optional internal detail of the flasks shown in FIGS. 1 to 3.

### DETAILED DESCRIPTION

The disclosure, together with its objects and the advantages thereof, may be understood better by reference to the following description taken in conjunction with the accompanying drawings, in which like reference numerals identify like elements in the drawings.

An initial seed cell culture has a relatively low volume, for example, about 10 ml or lower. Without being bound by theory, proximity of cells in the culture may be beneficial to growth. Accordingly, a low volume initial seed culture may benefit from being introduced into a container having a relatively smaller volume at initial stages of growth, followed by successive transfer(s) to higher volume container(s) after growth. However, such transfers result in increased costs, increased process complexity, and increased risk of contamination.

While containers such as flasks may be used instead of bags, conventional flasks also require multiple transfers of seed volumes in different containers. Moreover, in conventional flasks, as the cell culture grows and expands, the upper meniscus of the culture reduces in surface area, which gradually reduces oxygenation of the cell culture through the surface of the upper meniscus.

Flasks according to the disclosure avoid or reduce the need for transfer and provide better oxygenation. In some embodiments, a bioreactor flask includes a volume extending between a first volume end and a second volume end for the cultivation of cells or other microorganisms. The volume has a horizontal cross section area (CSA) which increases in at least a portion in a direction from the first volume end to the second volume end. In some embodiments, the second volume end is gravitationally upward relative to the first volume end.

The increase in the CSA of the container accommodates the growing volume of the cell culture while maintaining cell proximity. Further, the upper meniscus increases in surface area in at least an upper portion of the flasks, for example, with the increasing horizontal CSA, thereby increasing oxygenation through the surface of the upper meniscus as the cell culture grows.

In some embodiments, a bioreactor flask includes one or more internal structures to promote mixing and oxygenation in response to agitation of the flask, for example, by an agitation table. The internal structures may include baffles or ribs that protrude inwards of the flask.

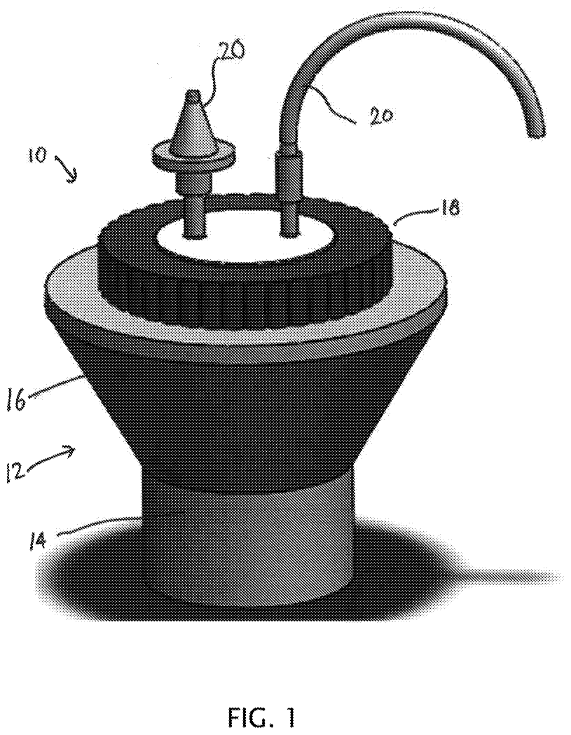

Referring to FIG. 1, there is shown a bioreactor flask 10 comprising a container 12 having a housing 11. The housing 11 may be formed of a material including at least one of a polymer, a glass, a metal, or an alloy, or a composite. In some embodiments, housing 11 is formed of a material that includes a polymer. In some embodiments, housing 11 is formed of a material that consists essentially of the polymer. In some embodiments, the material is rigid, for example, resisting deformation in response to applied force or pressure. In other embodiments, the material is flexible, for example, exhibiting partial elastic deformation in response to applied force or pressure, and tending to resume an original shape or configuration after the applied force or pressure is removed.

In some embodiments, the housing 11 is unitary. For example, the housing 11 is integrally formed, for example, by molding, stamping, casting, machining, or any other suitable process, so that each component of housing 11 is internally free of interfaces. In some embodiments, the housing 11 is formed by joining, welding, attaching, adhering, or unifying multiple components.

In some embodiments, the housing 11 includes a transparent or translucent window configured to indicate at least a portion of the contents of the volume. In other embodiments, the housing 11 is substantially opaque, for example, to shield or protect the contents of the volume from external light.

Housing 11 includes lower portion 14 and an upper portion 16. In some embodiments, upper portion 16 is formed as an inverted conical housing portion. In some examples, upper portion 16 is formed as an inverted truncated conical housing portion. The conical surface of upper portion 16 may define a predetermined angle relative to a vertical direction, for example, at least 30°, or at least 45°, or at least 60°. In some embodiments, upper portion 16 may be formed of a non-conical surface, for example, a curved or contoured surface, that gradually widens or otherwise exhibits an increase in CSA.

For example, the initial or seed culture may be introduced into the smaller volume of the lower portion 14, thus maintaining proximity and promoting culture growth, and as the cell culture grows, the culture may progressively expands into the higher volume of the upper portion 16.

In some embodiments, the geometric volume of the lower portion 14 is smaller than the geometric volume of the upper portion 16. In some embodiments, the geometric volume of the lower portion 14 is less than about 100%, or less than about 7%, or less than about 50%, or less than about 40%, or less than about 30%, or less than about 20%, or less than about 10%, or less than about 5%, or less than about 1%, of the volume of the upper portion 16. In some embodiments, the geometric volume of the lower portion 14 is less than about 20 ml, or less than about 10 ml, or less than about 5 ml, or less than about 2 ml, or less than about 1 ml. In some embodiments, the geometric volume of the upper portion 16 is greater than about 5 ml, or greater than about 10 ml, or greater than about 100 ml, or greater than about 200 ml, or greater than about 500 ml, or greater than about 1,000 ml, or greater than about 1,500 ml, or greater than about 2,000 ml. In some embodiments, the geometric volume of the lower portion 14 is about 10 ml, and the geometric volume of the upper portion 16 is about 2,000 ml. In some embodiments, the height of the lower portion 14 is less than about 100%, or less than about 75%, or less than about 50%, or less than about 40%, or less than about 30%, or less than about 20%, or less than about 10%, or less than about 5%, of the height of the upper portion 16.

It will be apparent from the external shape of the flask 10 that an initial culture volume is formed by the lower portion 14 or by a lower region of the upper portion 16 and as the volume of a culture increases that volume will occupy the increasing volume provided by the outwardly tapering upper housing 16. Thus, the bioreactor flask 10 includes a volume extending between a first volume end and a second volume end for the cultivation of cells or other microorganisms. The volume has a horizontal cross section area (CSA) which increases in a direction from the first volume end to the second volume end. In some embodiments, the second volume end is gravitationally upward relative to the first volume end. In some embodiments, the first volume end is between the second volume end and the cylindrical lower portion 16.

In some embodiments, the lower portion 14 is generally cylindrical. In other embodiments, the lower portion 14 may have any other suitable shape. In some embodiments, the lower portion 14 is conical, curved, or contoured. For example, the conicity of the lower portion 14 may be different from the conicity of the upper portion 16. In some embodiments, the lower portion 14 may narrow in a direction upward with respect to gravity, for example, along a portion of lower portion 14, or along an entirety of lower portion 14.

The lower portion 14 is optional. Thus, in some embodiments, housing 11 of flask 10 does not include lower portion 14, and only includes portion 16. In other embodiments, flask 10 includes the housing 11 including both the cylindrical lower portion 14 and the upper portion 16. Thus, the upper portion 16 defines the volume which provides said increasing CSA. In some embodiments, housing 11 may not include a separate lower portion 14, and a lower portion of upper portion 16 may function similar to lower portion 14. For example, the lower portion of upper portion 16 (relative to gravity) may have a smaller volume suitable for accommodating a relatively smaller seed or initial culture. In some embodiments, the lower portion of upper portion 16 may have a non-conical surface, or otherwise exhibit a same CSA, or reducing CSA, and only the upper portion of upper portion 16 may exhibit an increasing CSA.

In some embodiments, the CSA of upper portion 16 may increase non-monotically, or intermittently, or periodically, in an upward direction relative to gravity. For example, the CSA of upper portion 16 may increase in staggered portions or sections, like a ziggurat or stepped conical pyramid. In some embodiments, one or more portions of the upper portion 16 may exhibit a reduction in CSA, however, with upper portion 16 as a whole exhibiting an increase in CSA from a lower portion to an upper portion. Thus, while in some embodiments, the CSA of upper portion 16 may continuously increase from a lower end to an upper end of upper portion 16, in other embodiments, the CSA may increase non-continuously, or intermittently, or in a staggered or stepped manner.

While the lower portion 14 and the upper portion 16 may exhibit a ridge or angled intersection and an exterior and/or interior surface, in other embodiments, the lower portion 14 may smoothly transition to the upper portion 16.

Thus, in use, the flask shape provides an increasing volume for cells cultured therein, and particularly, an open surface area, the top surface of liquids in the volume which increases with increasing cell culture liquid volume, so that oxygen can more readily dissolve into the liquid.

In some embodiments, the bioreactor flask includes a fluid-tight lid 18. In some embodiments, the fluid-tight lid 18 is a screw-top lid. In some embodiments, the fluid-tight lid 18 is adjacent the second volume end.

In some embodiments, the bioreactor flask 10 includes at least one inlet and/or at least one outlet 20. In some embodiments, the flask 10 includes both the fluid-tight lid 18 and at least one inlet and/or at least one outlet 20. In some such embodiments, the at least one inlet and/or the at least one outlet 20 may extend through the lid 18.

In some embodiments, the flask 10 further includes for example, a screw top fluid-tight lid 18 and inlets/outlets 20.

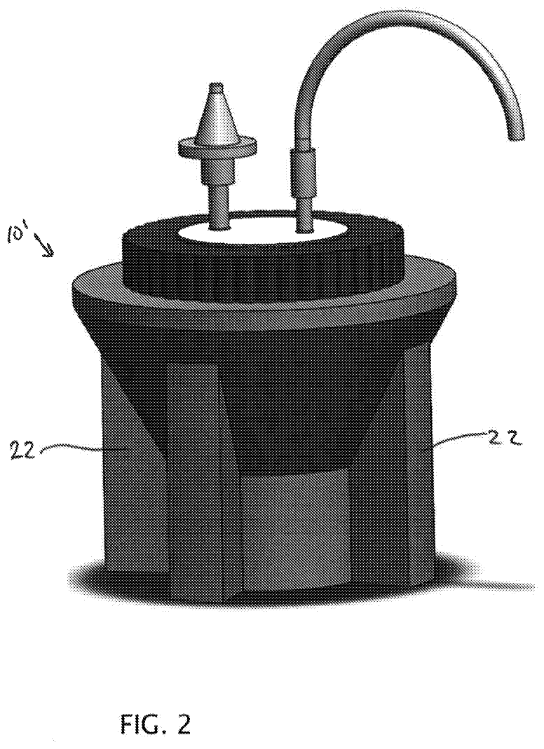

FIG. 2 shows a flask 10′ which is similar in construction to the flask 10, except that the flask 10′ further includes at least one leg 22 to stabilise the flask 10 in use, for example when it is resting on an agitation table (not shown). The at least one leg 22 may include one, two, three, four, or more legs. In some embodiments, legs 22 are symmetrically disposed about the flask 10′.

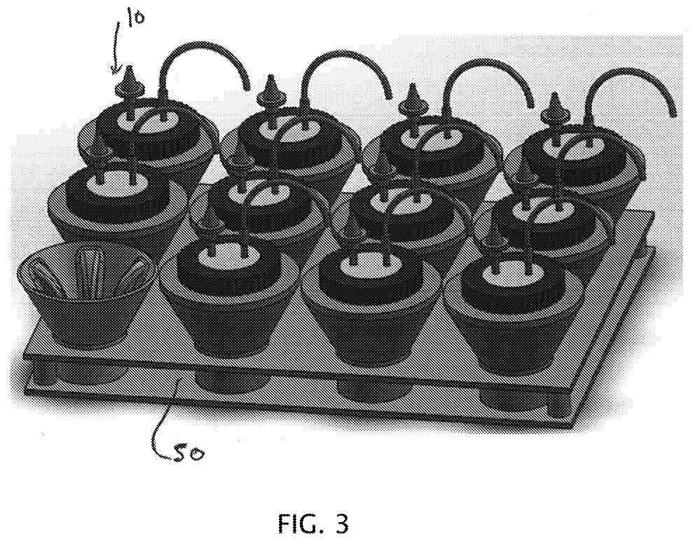

FIG. 3 shows an array of flasks 10 supported on a tray 50. In this embodiment, the tray can be mounted to a rockable bioreactor such that the array of flasks can be agitated together. While not shown in FIG. 3, at least one flask of the array of flasks may include at least one external stabilizing leg 22.

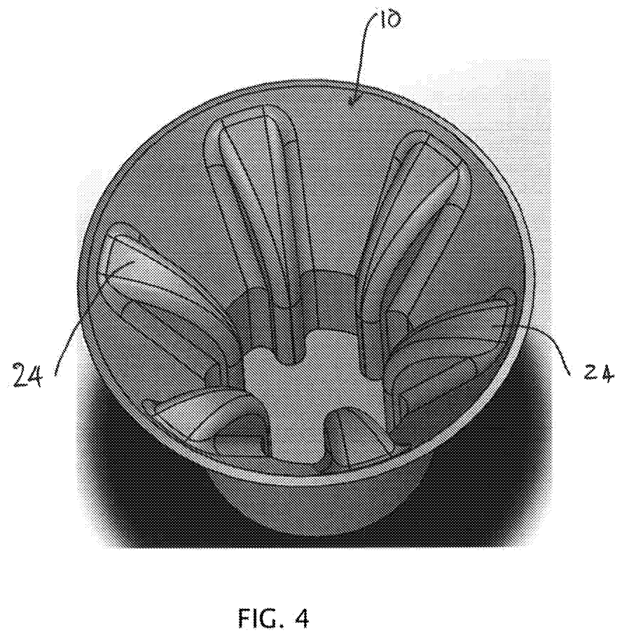

FIG. 4 shows one example of the internal structure of the flask 10 which includes one or more inwardly protruding ribs 24, to provide enhanced mixing of cells and a culture liquid in which cells are suspended during the agitation mentioned above. In this view the increasing volume of the flask can be more clearly seen. In some embodiments, each rib of the one or more inwardly protruding ribs 24 defines a lateral width that narrows in a portion extending in a direction away from the second volume end. In some embodiments, each rib of the one or more inwardly protruding ribs 24 defines a horizontal tapering protrusion that narrows in a portion extending in a direction toward the second volume end. Thus, the increase in the CSA can be further enhanced by upwardly reducing horizontal height or other dimensions or otherwise tapering of the ribs 24 as illustrated.

In some embodiments, each rib of the one or more inwardly protruding ribs 24 extends between the second volume end and the first volume end. For example, the ribs 24 may extend between ends of only upper portion 16. In other embodiments, the ribs 24 may extend from an end of upper portion 16 to an end of lower portion 14. For example, each rib of the one or more inwardly protruding ribs 24 may extend between the second volume end and a lower end of the cylindrical lower portion 14.

In some embodiments, each rib of the ribs 24 has substantially the same shape, size, and contour. In other embodiments, different ribs of the ribs 24 may differ in one or more of shape, size, or contour. For example, alternating ribs may differ in one or more of shape, size, or contour. The ribs 24 may include one, two, three, four, five, six, or more ribs, and may include even or odd numbers of ribs. The ribs 24 may be continuous, or be formed of sub-ribs or separated portions thereof.

Although embodiments have been described and illustrated, it will be apparent to the skilled addressee that additions, omissions and modifications are possible to those embodiments without departing from the scope of the invention claimed. For example, the ribs, legs, and the housing may be formed of the same material, or different materials. The ribs, legs, and the housing may be integrally formed or unitary, or may formed separately and joined. The inlet and/or the outlet may include one or more tubes extending through the lid, or otherwise into the volume. One or more of the inlet(s) or the outlet(s) may be adjacent an upper end of the flask, and others of the inlet(s) or the outlet(s) may be adjacent a lower end of the flask. The interior of the housing may be substantially smooth, for example, non-stick or low-friction, so that the cells may grow and expand in volume unimpeded. The exterior of the housing may be provided with a texture or pattern for facilitating holding or gripping of the flask.

1. A bioreactor flask comprising a volume extending between a first volume end and a second volume end for the cultivation of cells or other microorganisms, said volume having a horizontal cross section area (CSA) which increases in a direction in at least a portion from the first volume end to the second volume end.

2. The bioreactor flask of claim 1, wherein the second volume end is gravitationally upward relative to the first volume end.

3. The bioreactor flask of claim 2, wherein the bioreactor flask comprises a housing comprising a cylindrical lower portion and an inverted truncated conical upper portion, the inverted truncated conical upper portion defining the volume which provides said increasing CSA.

4. The bioreactor flask of claim 3, wherein the first volume end is between the second volume end and the cylindrical lower portion.

5. The bioreactor flask of claim 3, wherein the housing is formed of a material comprising at least one of a polymer, a glass, a metal, or an alloy.

6. The bioreactor flask of claim 3, wherein the housing is unitary.

7. The bioreactor flask of claim 3, wherein the housing comprises a transparent or translucent window configured to indicate at least a portion of the contents of the volume.

8. The bioreactor flask of claim 1, wherein the housing defines one or more inwardly protruding ribs.

9. The bioreactor flask of claim 8, wherein each rib of the one or more inwardly protruding ribs extends between the second volume end and the first volume end.

10. The bioreactor flask of claim 8, wherein each rib of the one or more inwardly protruding ribs extends between the second volume end and a lower end of the cylindrical lower portion.

11. The bioreactor flask of claim 8, wherein each rib of the one or more inwardly protruding ribs defines a lateral width that narrows in a portion extending in a direction away from the second volume end.

12. The bioreactor flask of claim 8, wherein each rib of the one or more inwardly protruding ribs defines a horizontal tapering protrusion that narrows in a portion extending in a direction toward the second volume end.

13. The bioreactor flask of claim 1, wherein the bioreactor flask comprises a fluid-tight lid.

14. The bioreactor flask of claim 13, wherein the fluid-tight lid is adjacent the second volume end.

15. The bioreactor flask of claim 1, wherein the bioreactor flask comprises at least one inlet and at least one outlet.

16. The bioreactor flask of claim 15, wherein the bioreactor flask comprises a fluid-tight lid, and wherein the at least one inlet and the at least one outlet extend through the lid.

17. The bioreactor flask of claim 16, wherein the fluid-tight lid is adjacent the second volume end.

18. The bioreactor flask of claim 1, wherein the bioreactor flask comprises at least one external stabilizing leg.

19. An array of flasks comprising at least one bioreactor flask of claim 1, wherein respective flasks of said array of flasks are supported in a tray for collective agitation.

20. The array of claim 19, wherein the at least one bioreactor flask comprises at least one external stabilizing leg.
